(19) United States
(12) Patent Application Publication
(10) Pub. No.: US 2007/0185438 A1
(43) Pub. Date: Aug. 9, 2007

(54) Multi-chamber mixing ampoule

(76) Inventors: Shlomo Haimi, Akiva (IL); Yehezkel Menashe, Petach Tikva (IL)

Correspondence Address: DR. MARK FRIEDMAN LTD.;c/o Bill Polkinghorn, 9003 Florin Way, Upper Marlboro, MD, 20772, US

(21) Appl. No.: 11/350,120

(22) Filed: Feb. 9, 2006

(51) Int. Cl. A61M 37/00

(52) U.S. Cl. 604/82

(57) ABSTRACT

The present invention provides a multi-compartment ampoule for the storage of the separated components of a multiple component mixture and then mixing them together in a single ampoule. The ampoule housing has a proximal dispensing end. The distal end of the ampoule housing is sealed by an end plug configured with an axial through bore. A portion of an axially displaceable plunger element passes thorough the axial through bore and extends into the interior volume and is fixedly attached to an axially displaceable piston element slideablely deployed within the ampoule housing so as to sealingly divide the interior volume into at least first and second storage regions that are pre-filled with the separate components that will be mixed together just prior to use. Axial displacement of the plunger element, and thereby the piston element, in a first direction discharges a first component of the mixture from the distal storage region through a one-way flow configuration into the proximal storage region. After the two components are in a single storage region, they may be thoroughly mixed together. Once the components are mixed, dispensing of the mixture may be by any number of methods. The mixture may be dispensed as if from a standard ampoule, wherein the needle of a syringe is inserted through the sealing membrane and the prescribed amount of the mixture is drawn out. Alternatively, the ampoule may be inserted into an injection device, such as an injection pen for example, in a manner similar to a standard ampoule. The additional features of a needle cannula configured for attachment directly to the proximal dispensing end of the ampoule housing and graduated markings on the shaft of the plunger that are indicative of volume dispensation combined with the basic ampoule provides an embodiment that can store the separated components of the mixture before use, provide a way of mixing the components together in the same ampoule and that can be used as the syringe to dispense the mixture directly into the patient.

### FIELD AND BACKGROUND OF THE INVENTION

The present invention relates to medical ampoules and, in particular, it concerns a multi-chambered ampoule configured to allow mixing of two or more substances just prior to use.

It is known to provide medical storage containers having multiple compartments in which the seal between the compartments is breached and the contents of the compartments are mixed just prior to use. These storage containers are particularly useful with medications whose unmixed shelf life is longer than its mixed shelf life. Some of these containers employ a method of puncturing or tearing a sealing membrane.

U.S. Pat. No. 6,981,963 to Barker et al. discloses a device for mixing and injecting medication from a two-chambered cartridge. A two-chambered cartridge is attached to a barrel and contains components of a medication stored separately in the chambers. A plunger in the rearward end of the cartridge can be advanced into the cartridge thereby forcing a piercing member to puncture a sealing membrane allowing the separate components to combine. As the cartridge is advanced forwardly into the barrel, the medication is injected through a needle and into a patient. The Barked device suffers from complexity of design and manufacture and does not allow for dispensing of the combined components into a standard syringe or with another dispensing device.

Another method of sealing compartments is to provide a movable partition. U.S. Pat. No. 6,113,257 to Sharon et al. discloses a container holding two separate components which are mixed prior to use. The container includes a housing with a dispensing opening and neck portion between the opening and the bottom end of the housing. The container further includes a displaceable member disposed within the housing and having a partition wall with an edge adapted for a sealing engagement with internal walls of the neck portion, thereby defining two compartments separated by the partition wall. The displaceable member is axially displaceable between two positions along an axis extending through the neck portion. These two positions comprise a first position where the edge of the partition wall sealingly engages the internal walls of the neck portion, and a second position where the edge of the partition wall and the internal walls are disengaged permitting flow communication between the two compartments. This flow communication allows mixing of the two components to form a formulation which is then ready for use. The Sharon et al. device is intended for such mixtures as infant formula and the like and is not well suited for medical applications where the container is fitted into an injection device.

There is therefore a need for a multi-chambered ampoule configured to allow mixing of two or more substances just prior to use that has a simple design and flexible dispensing modes.

### SUMMARY OF THE INVENTION

The present invention is a multi-chambered ampoule configured to allow mixing of two or more substances just prior to use that has a simple design and flexible dispensing modes.

According to the teachings of the present invention there is provided, a multi-compartment ampoule for the storage and mixing of multiple components of a mixture, the ampoule comprising: a) an ampoule housing defining an interior volume, said ampoule housing having a initially sealed proximal end; b) an end plug configured to seal a distal end of said ampoule housing, said end plug having an axial through bore; c) an axially displaceable plunger element configured such that a portion of said plunger element passes thorough said axial through bore into said interior volume and is fixedly attached to at least a first axially displaceable piston element slideablely deployed within said ampoule housing so as to sealingly divide said interior volume into at least first and second storage regions; and d) at least a first one-way flow configuration configured such that axial displacement of said plunger element in a first direction discharges a first component of the mixture from said first storage region through said one-way flow configuration into said second storage region so as to mix with at least a second component of the mixture being stored therein.

According to a further teaching of the present invention, said ampoule housing and said axially displaceable piston element have corresponding substantially circular cross-sectional contours.

According to a further teaching of the present invention, at least a portion of said one-way flow configuration is configured in said plunger element.

According to a further teaching of the present invention, at least said second component of the mixture is in a solid state.

According to a further teaching of the present invention, at least said second component of the mixture is in a powdered state.

According to a further teaching of the present invention, said plunger element is configured with at least one indicator of volume dispensation corresponding to axial displacement of said axially displaceable piston element.

According to a further teaching of the present invention, dispensing of the mixture effects axial displacement of said plunger element in a second direction.

According to a further teaching of the present invention, there is also provided, an adjustable displacement limitation element configured to limit said axial displacement of said plunger element in said second direction, thereby limiting an amount of the mixture dispensed.

According to a further teaching of the present invention, axial displacement of said plunger element in a second direction effects dispensing of the mixture.

According to a further teaching of the present invention, there is also provided, a needle cannula configured for attachment to said proximal dispensing end of said ampoule housing.

According to a further teaching of the present invention, there is also provided, an adjustable displacement limitation element configured to limit said axial displacement of said plunger element in said second direction, thereby limiting an amount of the mixture dispensed.

According to a further teaching of the present invention, said one-way flow configuration includes a one-way valve.

According to a further teaching of the present invention, at least one of said first and said second storage regions are sealed in a vacuum state.

There is also provided according to the teachings of the present invention, a method for storing multiple components of a mixture in separate compartments of an ampoule, then combining and mixing the components prior to use, the method comprising: a) providing a multi-compartmental ampoule having: i) an ampoule housing having an interior volume, said interior volume being divided into at least first and second storage regions by at least a first axially displaceable piston element slideablely deployed within said ampoule housing; and ii) at least a first one-way flow configuration configured such that axial displacement of said axially displaceable piston element in a first direction discharges a first component of the mixture from said first storage region through said one-way flow configuration into said second storage region so as to mix with at least a second component of the mixture being stored therein; and b) axially displacing said axially displaceable piston element in a first direction so as to discharge said first component of the mixture from said first storage region through said one-way flow configuration into said second storage region so as to mix with at least said second component of the mixture.

According to a further teaching of the present invention, there is also provided, providing an axially displaceable plunger element configured such that a portion of said plunger element extends into said interior volume and is fixedly attached to said axially displaceable piston element such that axial displacement of said plunger element affects axial displacement of said piston: element.

There is also provided according to the teachings of the present invention, a method for storing, mixing and dispensing multiple components of a mixture from an ampoule having separate storage compartments, the method comprising: a) providing a multi-compartmental ampoule having: i) an ampoule housing defining an interior volume, said ampoule housing having a proximal dispensing end; ii) an axially displaceable plunger element configured such that a portion of said plunger element extends into said interior volume and is fixedly attached to at least a first axially displaceable piston element slideablely deployed within said ampoule housing so as to sealingly divide said interior volume into at least first and second storage regions; and iii) at least a first one-way flow configuration configured such that axial displacement of said plunger element in a first direction discharges a first component of the mixture from said first storage region through said one-way flow passage into said second storage region so as to mix with at least a second component of the mixture being stored therein; b) axially displacing said axially displaceable piston element in a first direction so as to discharge said first component of the mixture from said first storage region through said one-way flow configuration into said second storage region so as to mix with at least said second component of the mixture; and c) axially displacing said axially displaceable piston element in a second direction so as to dispense the mixture.

According to a further teaching of the present invention, said ampoule housing defining is implemented as substantially a cylinder.

According to a further teaching of the present invention, said plunger element is implemented with at least one indicator of volume dispensation corresponding to axial displacement of said axially displaceable piston element.

According to a further teaching of the present invention, there is also provided: a) providing a needle cannula configured for attachment to said proximal dispensing end of said ampoule housing; and b) attaching said needle cannula to said proximal dispensing end of said ampoule housing.

According to a further teaching of the present invention, there is also provided, providing an adjustable displacement limitation element configured to limit said axial displacement of said plunger element in said second direction, thereby limiting an amount of the mixture dispensed.

### BRIEF DESCRIPTION OF THE DRAWINGS

The invention is herein described, by way of example only, with reference to the accompanying drawings, wherein:

FIG. 1 is longitudinal cross-sectional view of a preferred embodiment of a syringe constructed and operative according to the teachings of the present invention, showing the beginning of the combining process;

FIG. 2 is longitudinal cross-sectional view of the embodiment of FIG. 1 as the combining process continues:

FIG. 3 is longitudinal cross-sectional view of the embodiment of FIG. 1 at the completion of the combining process;

FIG. 4 is a detail of the embodiment of FIG. 1 showing a preferred embodiment of a piston and one-way flow passage constructed and operative according to the teachings of the present invention;

FIG. 5 is longitudinal cross-sectional view of a preferred embodiment of an ampoule constructed and operative according to the teachings of the present invention, shown with the components of the mixture in a storage state;

FIG. 6 is longitudinal cross-sectional view of the embodiment of FIG. 5 shown with the components of the mixture in a mixed state.

### DESCRIPTION OF THE PREFERRED EMBODIMENTS

The present invention is a multi-chambered ampoule configured to allow mixing of two or more substances just prior to use that has a simple design and flexible dispensing modes.

The principles and operation of a multi-chambered ampoule according to the present invention may be better understood with reference to the drawings and the accompanying description.

By way of introduction, the present invention provides a multi-compartment ampoule for the storage of the separated components of a multiple component mixture and then mixing them together in a single ampoule. This provides a method of storing the separate components in a manner that will provide the longest shelf life for the components. It also provides a sterile mixing environment. A further advantage is that the mixing process may be performed by personnel with lower technical skills than are currently required for such a task. This is of particular benefit for use in situation where a sterile environment is not available such as in the field for example. Although the embodiments described herein are configured with two separate compartments, this is not intended as a limitation, and it should be understood that embodiment having more than two compartments are within the scope of the present invention.

The ampoule housing defines an interior volume, and is initially sealed at its proximal end by substantially any known arrangement such as, but not limited to, a cap and any ampoule sealing membrane known in the art. The distal end of the ampoule housing is sealed by an end plug configured with an axial through bore. A portion of an axially displaceable plunger element passes thorough the axial through bore and extends into the interior volume and is fixedly attached to an axially displaceable piston element slideablely deployed within the ampoule housing so as to sealingly divide the interior volume into at least first and second storage regions that are pre-filled with the separate components that will be mixed together just prior to use. It will be appreciated that at least one of the components may be stored in a solid state, such as a powder. Alternatively, all of the components may be liquids. Axial displacement of the plunger element, and thereby the piston element, in a first direction discharges a first component of the mixture from the distal storage region through a one-way flow passage into the proximal storage region. Therefore, the volume of the storage regions varies as the piston is displaced within the interior volume of the ampoule housing.

Once the two components are in a single storage region, they may be thoroughly mixed together. It will be appreciated that the one-way flow passage may be configured using substantially any one of the numerous methods known in the art such as, but not limited to, one-way ball valves, and flap valves, for example. It will be understood that the one-way flow passage is configured to be normally closed having an opening threshold pressure differential great enough to maintain the seal between the two storage regions during shipping and storage. It will be appreciated that any of the storage regions may be sealed in a state of vacuum. In such an embodiment, the opening threshold pressure differential of the one-way flow passage is great enough to withstand the associated vacuum pressure.

It should be noted that although the preferred embodiments described herein relate to ampoules having a substantially cylindrical body, this is not intended as a limitation and substantially any suitable cross-sectional contour is within the scope of the present invention; therefore, the term “axial” is used herein with regard to a direction of motion and is not intended to suggest or imply any geometrical shape to any of the structural elements of the present invention.

Once the components are mixed, dispensing of the mixture may be by any number of methods. The mixture may be dispensed as if from a standard ampoule, wherein the needle of a syringe is inserted through the sealing membrane and the prescribed amount of the mixture is drawn out. Alternatively, the ampoule may be inserted into an injection device, such as an injection pen for example, in a manner similar to a standard ampoule.

Further features of the present invention are to provide a needle cannula configured for attachment directly to the proximal dispensing end of the ampoule housing and to provide graduated markings on the shaft of the plunger that are indicative of volumetric dispensation corresponding to the axial displacement of the piston element. The combination of these features with the basic ampoule of the present invention provides an embodiment that can store the separated components of the mixture before use, provide a way of mixing the components together in the same ampoule and that can be used as the syringe to dispense the mixture directly into the patient. It should be noted that this syringe embodiment is of particular benefit in the areas of emergency and combat field medicine.

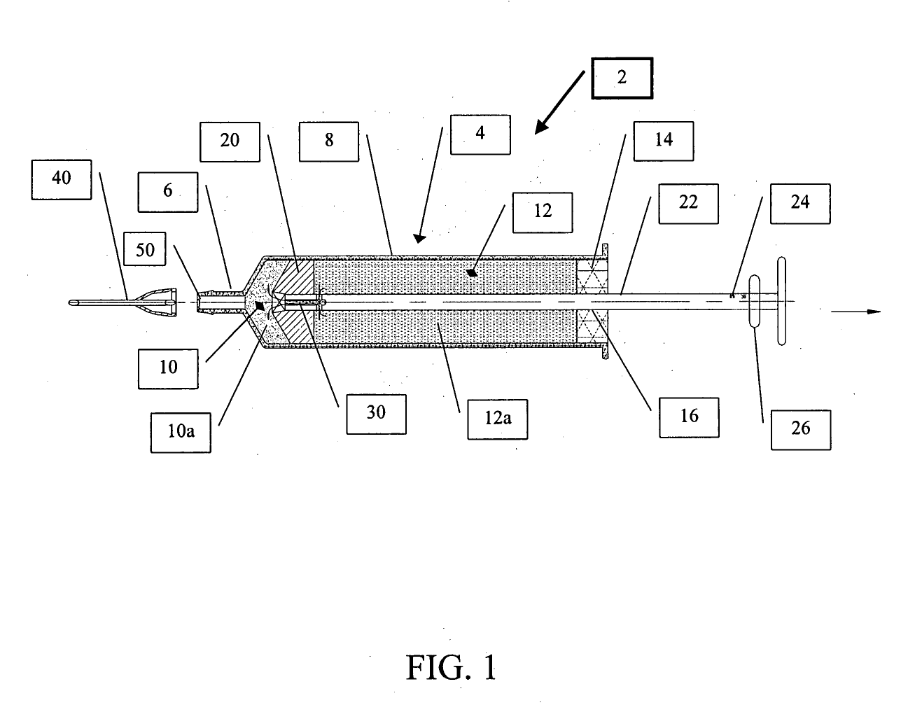

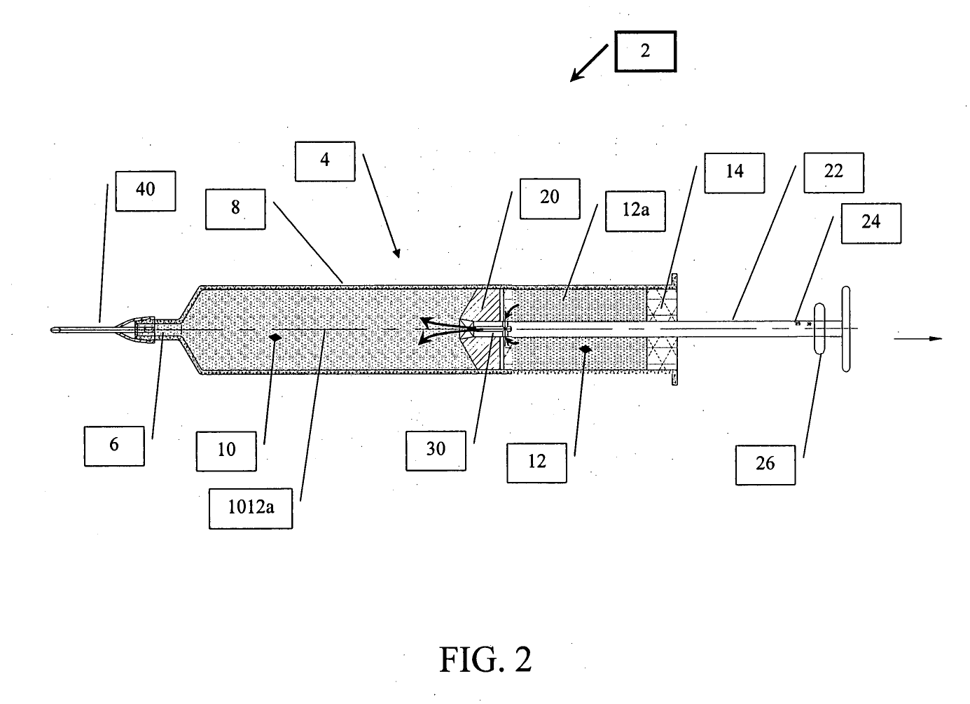

Referring now to the drawings, FIGS. 1-4 illustrate a first preferred embodiment 2 that may be used primarily as a syringe. The syringe housing 4 is configured with a proximal dispensing end 6 and a substantially cylindrical body 8. The proximal dispensing end 6 may be sealed by a removable cap or, as illustrated here, an ampoule sealing membrane 50 as is known in the art. The syringe housing 4 is sealed at its distal end by an end plug 14, which is configured with an axial through bore 16. The interior volume of the body 8 is divided into first 10 and second 12 storage regions by the axially displaceable piston element 20. Each of the storage regions 10 and 12 contain different components 10a and 12a respectively that are to later be combined and mixed together into the mixture referred to as 1012a. Component 12a is preferably a liquid. As mentioned above, either of the first 10 and second 12 storage regions may be sealed in a vacuum state. This may be of particular benefit regarding components of a mixture that deteriorate with prolonged exposure to the atmosphere, even in a closed container.

The plunger element 22 extends through the through bore 16 into the interior volume of the body 8 and is fixedly attached to the axially displaceable piston element 20. Therefore, axial displacement of the plunger element 22 affects axial displacement of the axially displaceable piston element 20.

In this embodiment, the one-way flow passage 30 is configured in the proximal end of the plunger element 22. It will be understood that one or more one-way flow passages may alternatively be configured elsewhere such as in the axially displaceable piston element 20, for example. In such an embodiment, the piston is configured as an element that allows through flow of the contents of the second storage region 12 when displaced in the first direction, and is sealed and applies pressure to the mixed contents of the first storage region 10 when displaced in the second direction.

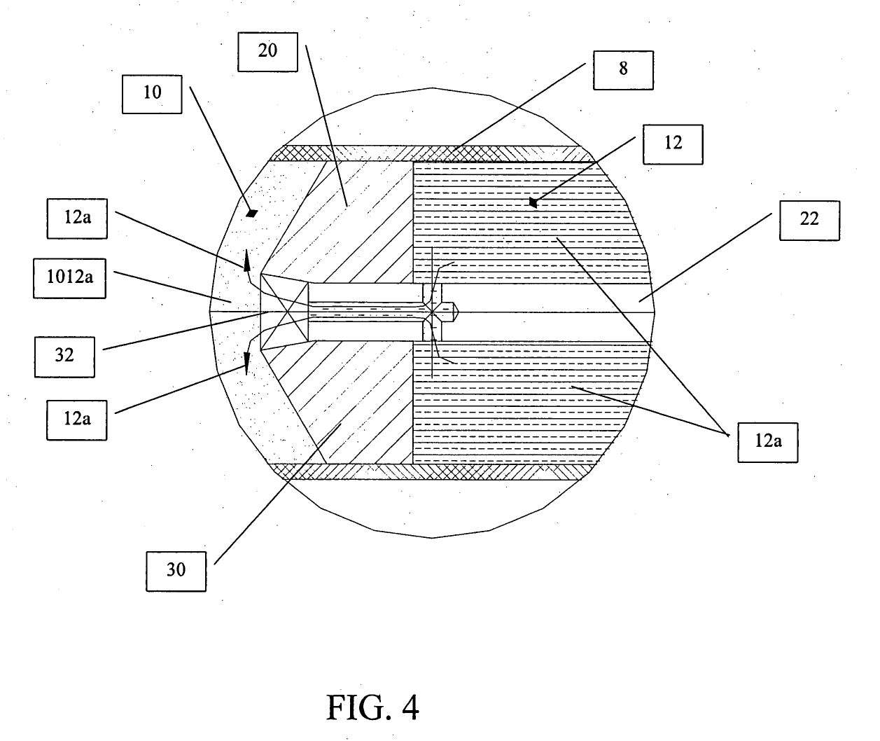

As seen in better detail in FIG. 4, as the plunger element 22, and consequently the axially displaceable piston element 20, is axially displaced in a distal direction, component 12a is discharged from storage region 12 through the one-way flow passage 30 and into storage region 10, there to be combined and mixed with component 10a. The distal end 32 of the one-way flow passage 30 may optionally be configured as a nozzle that generates turbulence as component 12a is discharged into storage region 10. FIG. 4 illustrates the syringe as the discharge process continues throughout the full distal stroke of the plunger element 22, thereby combining and mixing the components 10a and 12a to produce the desired mixture 1012a.

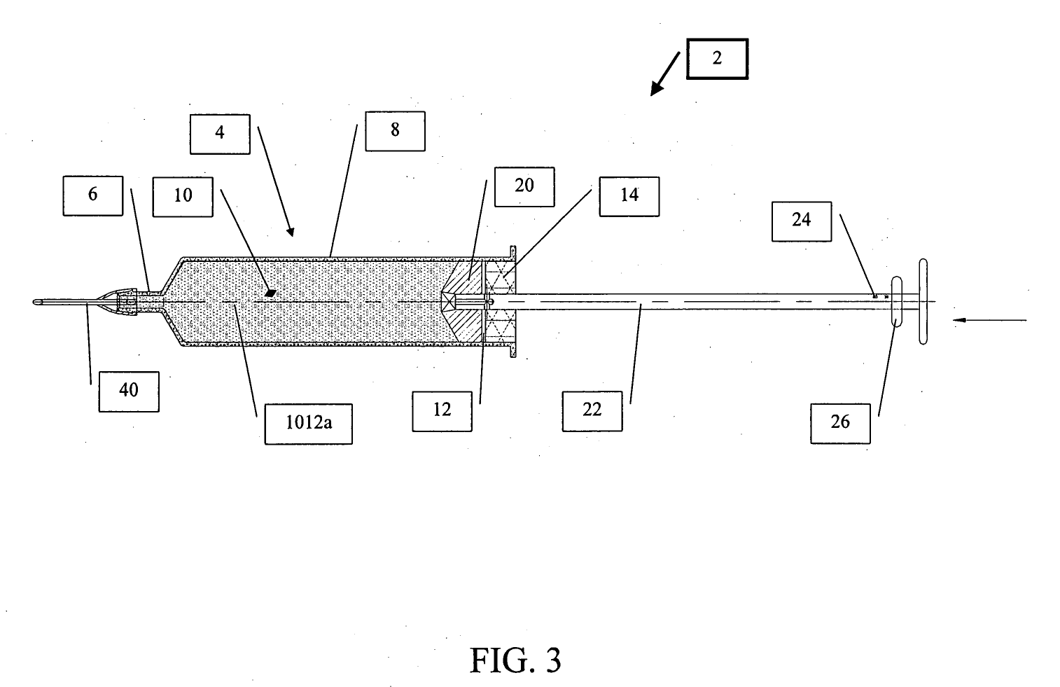

As illustrated in FIG. 3, once the mixing process in complete and the needle cannula 40 is attached to the proximal end 6 of the syringe housing 4, the syringe is ready to dispense the now prepared mixture 1012a as prescribed. Since, the one-way flow passage 30 is configured to allow only the contents of storage region 12 to flow in to storage region 10 as the plunger element 22, and thereby the piston element 20, is axially displaced in a proximal direction, the mixture 1012a is dispended through the needle cannula 40. The graduated markings 24 on the shaft of the plunger element 22 that are indicative of volumetric dispensation of the mixture 1012a corresponding to the axial displacement of the piston element 20. To ensure precise delivery of the prescribed amount of the mixture 1012a, the adjustable displacement limitation element 26, which is configured to limit the axial displacement of the plunger element 22 in the proximal direction, may be moved along the shaft of the plunger element 22 to the appropriate marking and thereby limit the amount of the mixture 1012a dispensed.

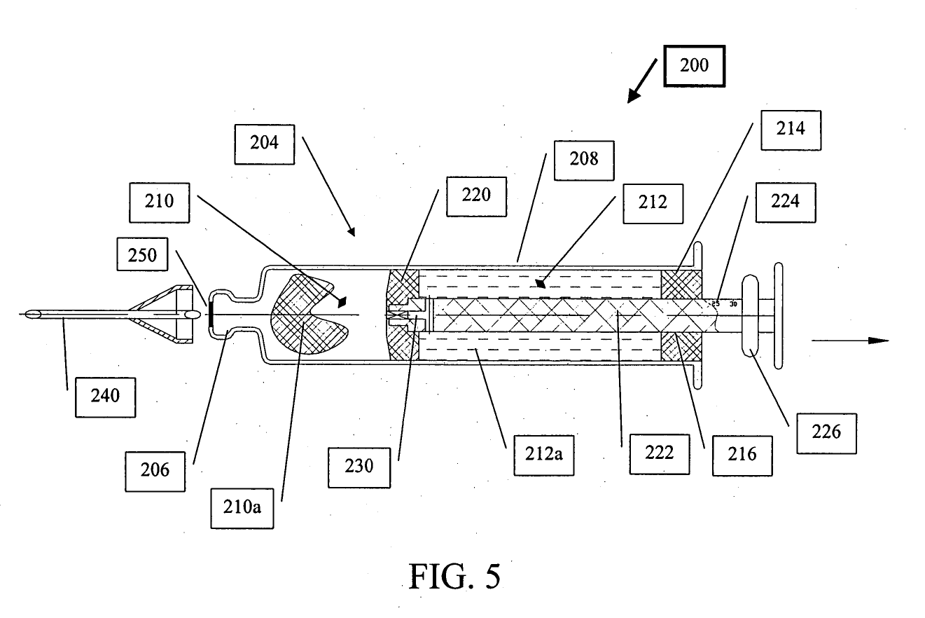

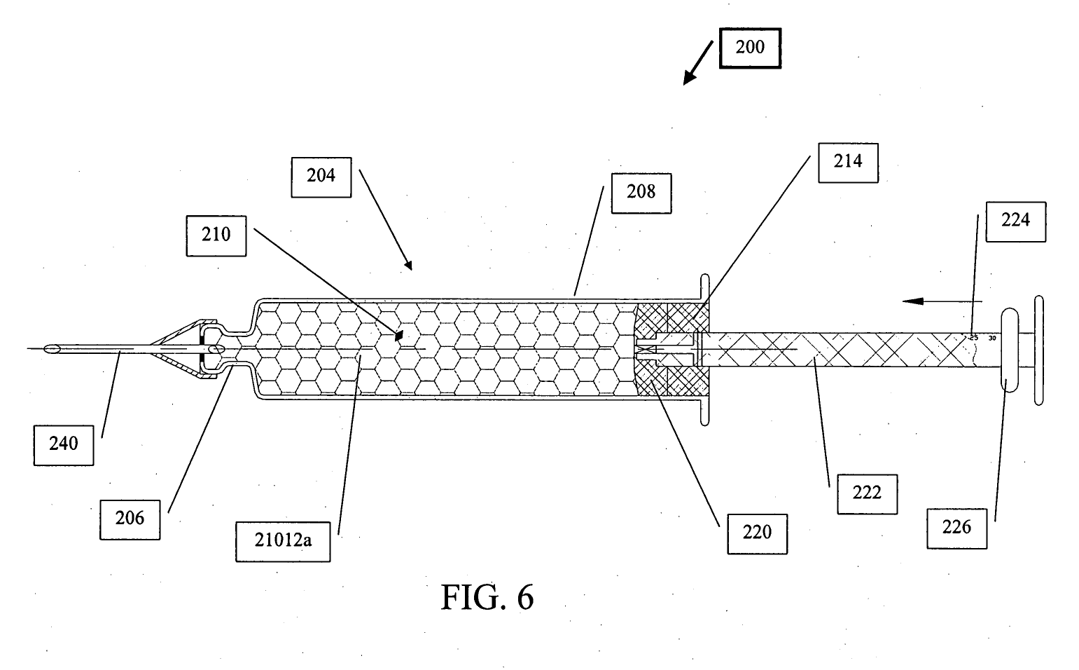

FIGS. 5 and 6 illustrate a second preferred embodiment 200 of the present invention that may be used as a syringe or as a standard ampoule, as discussed above. For ease of understanding, the elements of this embodiment are number similarly to the corresponding elements of embodiment 2 such that all reference numbers are increased by 200; therefore, the syringe housing 4 of embodiment 2 becomes the ampoule housing 204 of embodiment 200 and so on.

The operation of embodiment 200 is substantially the same as that of embodiment 2, the main difference being the shape of the proximal end 206 of the ampoule housing 204. Here the proximal end 206 is shaped substantially the same as a standard medical ampoule. Therefore, this embodiment of the present invention may be used in substantially the same manner as a standard medical ampoule. That is, embodiment 200 may be used to fill a standard medical syringe. In such a case, the vacuum created by drawing the mixture 21012a out of storage region 210 draws the piston element 220, and therefore the plunger element 222, is a proximal direction. In order to ensure that the prescribed amount of the mixture 21012a is precisely dispensed, the adjustable displacement limitation element 226 may be slidingly adjusted to the appropriate graduated markings 224 on the shaft of the plunger element 222 and thereby limit the amount of the mixture 21012a dispensed.

Alternatively, the ampoule of embodiment 200 may be used with an appropriately configured injection device.

This embodiment, as illustrated in FIG. 6, may also have a needle cannula 240 attached to the distal end 206 of the ampoule housing 204 and the mixture may be delivered directly to the patient.

It should be noted that the principles of the present invention need not be limited to medical devices and may be applied with equal benefit in regard to numerous industrial and chemical applications. Therefore, it will be appreciated that the above descriptions are intended only to serve as examples and that many other embodiments are possible within the spirit and the scope of the present invention.

What is claimed is:

1. A multi-compartment ampoule for the storage and mixing of multiple components of a mixture, the ampoule comprising:
(a) an ampoule housing defining an interior volume, said ampoule housing having a initially sealed proximal end;
(b) an end plug configured to seal a distal end of said ampoule housing, said end plug having an axial through bore;
(c) an axially displaceable plunger element configured such that a portion of said plunger element passes thorough said axial through bore into said interior volume and is fixedly attached to at least a first axially displaceable piston element slideablely deployed within said ampoule housing so as to sealingly divide said interior volume into at least first and second storage regions; and
(d) at least a first one-way flow configuration configured such that axial displacement of said plunger element in a first direction discharges a first component of the mixture from said first storage region through said one-way flow configuration into said second storage region so as to mix with at least a second component of the mixture being stored therein.

2. The ampoule of claim 1, wherein said ampoule housing and said axially displaceable piston element have corresponding substantially circular cross-sectional contours.

3. The ampoule of claim 1, wherein at least a portion of said one-way flow configuration is configured in said plunger element.

4. The ampoule of claim 1, wherein at least said second component of the mixture is in a solid state.

5. The ampoule of claim 4, wherein at least said second component of the mixture is in a powdered state.

6. The ampoule of claim 1, wherein said plunger element is configured with at least one indicator of volume dispensation corresponding to axial displacement of said axially displaceable piston element.

7. The ampoule of claim 1, wherein dispensing of the mixture effects axial displacement of said plunger element in a second direction.

8. The ampoule of claim 7, further including an adjustable displacement limitation element configured to limit said axial displacement of said plunger element in said second direction, thereby limiting an amount of the mixture dispensed.

9. The ampoule of claim 1, wherein axial displacement of said plunger element in a second direction effects dispensing of the mixture.

10. The ampoule of claim 9, further including a needle cannula configured for attachment to said proximal dispensing end of said ampoule housing.

11. The ampoule of claim 10, further including an adjustable displacement limitation element configured to limit said axial displacement of said plunger element in said second direction, thereby limiting an amount of the mixture dispensed.

12. The ampoule of claim 1, wherein said one-way flow configuration includes a one-way valve.

13. The ampoule of claim 1, wherein at least one of said first and said second storage regions are sealed in a vacuum state.

14. A method for storing multiple components of a mixture in separate compartments of an ampoule, then combining and mixing the components prior to use, the method comprising:
(a) providing a multi-compartmental ampoule having:
(i) an ampoule housing having an interior volume, said interior volume being divided into at least first and second storage regions by at least a first axially displaceable piston element slideablely deployed within said ampoule housing; and
(ii) at least a first one-way flow configuration configured such that axial displacement of said axially displaceable piston element in a first direction discharges a first component of the mixture from said first storage region through said one-way flow configuration into said second storage region so as to mix with at least a second component of the mixture being stored therein; and
(b) axially displacing said axially displaceable piston element in a first direction so as to discharge said first component of the mixture from said first storage region through said one-way flow configuration into said second storage region so as to mix with at least said second component of the mixture.

15. The method of claim 14, further including providing an axially displaceable plunger element configured such that a portion of said plunger element extends into said interior volume and is fixedly attached to said axially displaceable piston element such that axial displacement of said plunger element affects axial displacement of said piston element.

16. A method for storing, mixing and dispensing multiple components of a mixture from an ampoule having separate storage compartments, the method comprising:
(a) providing a multi-compartmental ampoule having:
(i) an ampoule housing defining an interior volume, said ampoule housing having a proximal dispensing end;
(ii) an axially displaceable plunger element configured such that a portion of said plunger element extends into said interior volume and is fixedly attached to at least a first axially displaceable piston element slideablely deployed within said ampoule housing so as to sealingly divide said interior volume into at least first and second storage regions; and
(iii) at least a first one-way flow configuration configured such that axial displacement of said plunger element in a first direction discharges a first component of the mixture from said first storage region through said one-way flow passage into said second storage region so as to mix with at least a second component of the mixture being stored therein;
(b) axially displacing said axially displaceable piston element in a first direction so as to discharge said first component of the mixture from said first storage region through said one-way flow configuration into said second storage region so as to mix with at least said second component of the mixture; and
(c) axially displacing said axially displaceable piston element in a second direction so as to dispense the mixture.

17. The method of claim 16, wherein said ampoule housing defining is implemented as substantially a cylinder.

18. The method of claim 16, wherein said plunger element is implemented with at least one indicator of volume dispensation corresponding to axial displacement of said axially displaceable piston element.

19. The method of claim 16, further including:
(a) providing a needle cannula configured for attachment to said proximal dispensing end of said ampoule housing; and
(b) attaching said needle cannula to said proximal dispensing end of said ampoule housing.

20. The method of claim 19, further including providing an adjustable displacement limitation element configured to limit said axial displacement of said plunger element in said second direction, thereby limiting an amount of the mixture dispensed.
